(19) United States
(12) Patent Application Publication
(10) Pub. No.: US 2016/0015552 A1
(43) Pub. Date: Jan. 21, 2016

(54) CRANIAL DEFORMATION CORRECTION HELMET AND METHOD FOR PRODUCING SAME

(71) Applicants: ONO & CO., LTD., Tokyo (JP); Ai-met NEO CO., Ltd., Tokyo (JP)

(72) Inventors: Hidenori Ono, Tokyo (JP); Yasuo Aihara, Tokyo (JP); Hisayuki Sugiyama, Kanagawa (JP); Toru Matsumoto, Tokyo (JP); Kazumi Kai, Tokyo (JP)

(73) Assignees: ONO & CO., LTD, Tokyo (JP); Ai-met NEO Co., Ltd., Tokyo (JP)

(21) Appl. No.: 14/771,792

(22) PCT Filed: Feb. 27, 2014
(86) PCT No.: PCT/JP2014/054913

§ 371 Date: Aug. 31, 2015

(30) Foreign Application Priority Data
Mar. 1, 2013 (JP) 2013-040509

(51) Int. Cl. A61F 5/058

(52) U.S. Cl. CPC A61F 5/05891

(57) ABSTRACT

A cranial deformation correction helmet (2) having a shell (4), which retains necessary strength and hardness, but can be sufficiently lightweight, and a method capable of producing such a cranial deformation correction helmet (2) with sufficient rapidity and at low cost, are provided.

To produce the helmet (2), a non-foamed synthetic resin shell (4) is shaped by selective laser sintering, and then a foamed synthetic resin liner (6) is disposed on the inner surface of the shell (4). The relative density of the shell (4) is 90 to 98%.

### TECHNICAL FIELD

This invention relates to a cranial deformation correction helmet for use in correcting cranial deformation in an infant, and a method for producing such a cranial deformation correction helmet.

### BACKGROUND ART

Cranial deformation in an infant includes, for example, plagiocephaly (a deformed shape in which the skull is not bilaterally symmetric, but is greatly inclined to one side), brachycephaly (a deformed shaped in which the longitudinal dimension of the skull is markedly small), and dolichocephaly is deformed shaped in which the longitudinal dimension of the skull is markedly large). As a mode of treatment for such a cranial deformation, a method comprising capping the skull of the infant with a cranial deformation correction helmet to lead the growth of the skull into the direction of correction of deformation is adopted as is well known. As a cranial deformation correction helmet for use in this treatment mode, Patent Document 1 indicated below discloses a cranial deformation correction helmet equipped with a non-foamed synthetic resin outer shell, and a foamed synthetic resin liner disposed on the inner surface of the shell. The shell is formed with a plurality of ventilation through-holes.

### PRIOR ART DOCUMENTS

### Patent Documents

Patent Document 1: JP-A-2003-532433

### SUMMARY OF THE INVENTION

### Problems to be Solved by the Invention

According to experiences of the present inventors, however, the conventional cranial deformation correction helmet disclosed in Patent Document 1 involves the following problems to be solved: First, allowing the shell to retain necessary strength and hardness usually requires that the thickness of the shell be a considerable thickness (of the order of 10 to 15 mm). Because of this thickness, the cranial deformation correction helmet becomes heavyweight. Treatment for cranial correction is desired to start when an infant' s skull is ungrown, namely, at a relative early time after birth, for example, within 4 months after birth. At a relatively early stage after birth, however, the infant's neck is also immature, and it is not desirable to cap the infant's head with a heavyweight cranial deformation correction helmet. Secondly, the shell is desired to be individually formed into a required shape according to the shape of an individual infant's skull to be corrected. If so-called single unit customizing is adopted, however, manufacturing takes a lengthy time and the manufacturing cost is markedly high.

The present invention has been accomplished in the light of the above-mentioned facts. A first technical challenge facing the present invention is to provide a novel and improved cranial deformation correction helmet which enables a shell to be sufficiently lightweight, although the shell has necessary strength and hardness.

A second technical challenge to the present invention is to provide a method for producing the novel and improved cranial deformation correction helmet, which makes it possible to produce a cranial deformation correction helmet capable of rendering a shell sufficiently lightweight, despite its retained necessary strength and hardness, sufficiently promptly and at sufficiently low cost, even when a so-called single unit customizing procedure is adopted.

Means for Solving the Problems

As a result of in-depth studies and prototype experiments, the present inventors have found that the above-mentioned first technical challenge can be overcome, for example, by shaping a shell by selective laser sintering such that the relative density of the shell (density relative to the density of the same shell which, however, is solid and contains no voids) is 90 to 98%.

That is, according to an aspect of the present invention, there is provided, as a cranial deformation correction helmet for solving the above first technical challenge, a cranial deformation correction helmet comprising a non-foamed synthetic resin outer shell, and a foamed synthetic resin inner liner disposed on the inner surface of the shell.
wherein the relative density of the shell is 90 to 98%.

It is preferred that a thick-wailed reinforcing portion having an increased wall thickness be formed at the outer peripheral edge of the shell. Advantageously, a plurality of ventilation through-holes are formed in the shell. Also advantageously, the Shore D hardness of the shell is 70 to 85, and the ball rebound resilience (ASTM D3574) of the liner is 1% or less. It is preferred for the shell to be opened at a site thereof corresponding to the top of the skull and to be in an annular shape as a whole. The following are preferred embodiments: In the shell, a slit extending from the upper edge to the lower edge is formed. A protruding piece extending out from an inner part in the thickness direction is disposed at a side edge of the slit, while a recessed concavity corresponding to the protruding piece is disposed in an inner part in the thickness direction at the other side edge of the slit. Mutual coupling means to be coupled together separably are arranged on the face of the protruding piece and the bottom surface of the recessed concavity. When the protruding piece is positioned in the recessed concavity, and the protruding piece and the recessed concavity are coupled together by the mutual coupling means, regions on both sides of the slit on the surface of the shell continue smoothly.

The aforementioned second technical challenge to the present invention can be overcome by shaping the shell by selective laser sintering.

That is, according to another aspect of the present invention, there is provided, as a method for solving the above second technical challenge, a method for producing the cranial deformation correction helmet for solving the first technical challenge, comprising:
shaping the shell by selective laser sintering based on the outer shape of the skull to be corrected, the outer shape being derived from scan data on the skull to be corrected; and
then disposing the liner on the inner surface of the shell.

### Effects of the Invention

In the cranial deformation correction helmet of the present invention, the shell shaped from the non-foamed synthetic resin is not one which is solid and contains no voids, but one having a relative density of 90 to 98%. Thus, the shell retains necessary strength and hardness, but can be sufficiently lightweight. Particularly when the thick-walled reinforcing portion having an increased wall thickness is formed at the outer peripheral edge of the shell, sufficient strength can be retained.

According to the method of the present invention, the shell is shaped by selective laser sintering based on the outer shape of the skull to be corrected. Thus, it is possible to produce the cranial deformation correction helmet, whose shell can be sufficiently lightweight while retaining necessary strength and hardness, with sufficient rapidity and at low cost, without requiring a relatively expensive mold.

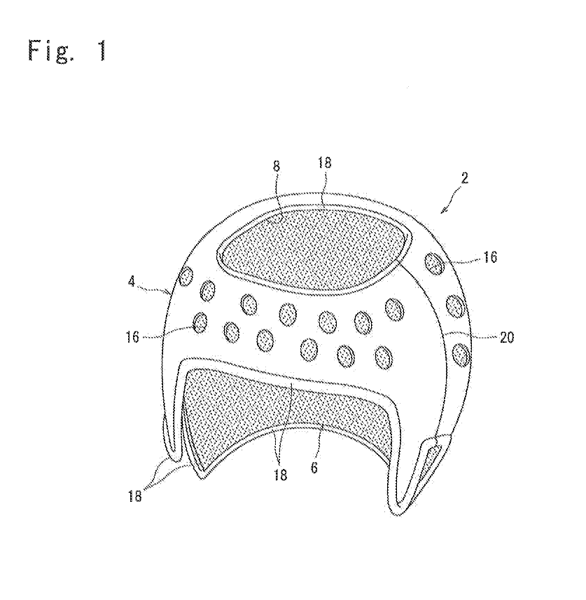

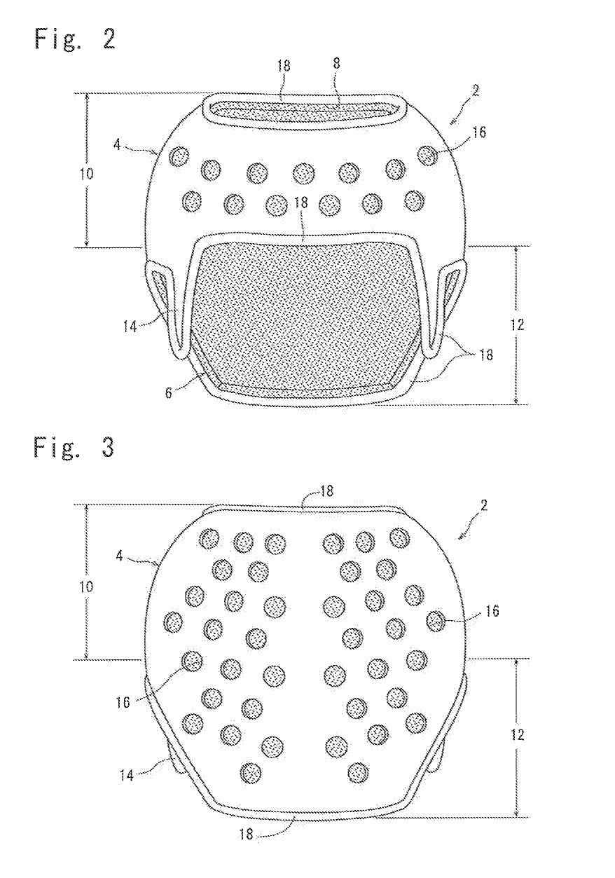

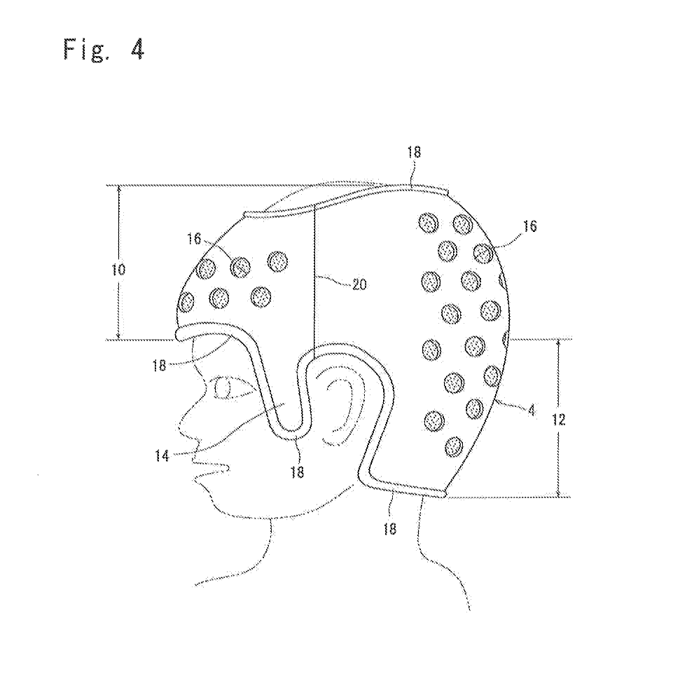

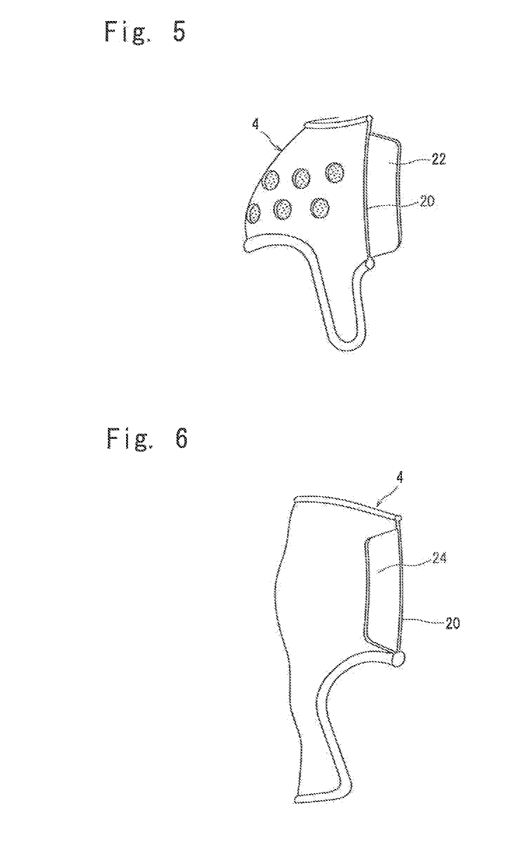

### BRIEF DESCRIPTION OF THE DRAWINGS

[FIG. 1] is a perspective view showing a preferred embodiment of a cranial deformation correction helmet configured in accordance with the present invention.

[FIG. 2] is a front view of the cranial deformation correction helmet in FIG. 1.

[FIG. 3] is a rear view of the cranial deformation correction helmet in FIG. 1.

[FIG. 4] is a right side view of the cranial deformation correction helmet in FIG. 1.

[FIG. 5] is a partial perspective view showing a protruding piece formed in a shell of the cranial deformation correction helmet in FIG. 1.

[FIG. 6] is a partial perspective view showing an accommodation concavity formed in the shell of the cranial deformation correction helmet in FIG. 1.

### MODE FOR CARRYING OUT THE INVENTION

The cranial deformation correction helmet configured in accordance with the present invention will be described in further detail by reference to the accompanying drawings showing its preferred embodiment.

Referring to FIG. 1, a cranial deformation correction helmet 2 illustrated there, which has been configured in accordance with the present invention, is composed of a non-foamed synthetic resin outer shell 4, and a foamed synthetic resin inner liner 6.

With further reference to FIGS. 2 to 4 along with FIG. 1, the shell 4 in the illustrated embodiment has a relatively large nearly circular opening 8 in an upper surface, is thus opened at a site corresponding to the top of the skull, and assumes an annular shape as a whole. In further detail, the shell 4 has a main portion 10 surrounding the peripheral edge of the skull, a back suspending portion 12 extending out downwardly from the back of the main portion 10, and protruding portions 14 protruding downwardly from both side surfaces of the main portion 10. As will be understood by referring to FIG. 4, the back suspending portion 12 is located opposite the back of a helmet wearer's neck, and the protruding portion 14 is located ahead of the helmet wearer's ear, so that the helmet wearer's ear is located between the back suspending portion 12 and the protruding portion 14. It is preferred that a plurality of ventilation through-holes 16 be formed in the shell 4. In the illustrated embodiment, the plurality of through-holes 16 are formed at suitable intervals in a front half and a rear half of the main portion 10 and in the back suspending portion 12. Each of the through-holes 16 is preferably a circular hole having a diameter of 5 to 15 mm.

Thick-walled reinforcing portions 18 having an increased wall thickness are preferably formed at the outer peripheral edges of the shell 4, namely, the nearly circular upper edge defining the opening 8, the lower edge of the main portion 10, and the free edges of the back suspending portion 12 and the protruding portions 14. The thick-walled reinforcing portion 18 may be a so-called circular edging whose cross-sectional shape is a nearly circular shape with a diameter of the order of 4 to 8 mm. The thickness of the shell 4, except the thick-walled reinforcing portion 18, may be of the order of 2 to 4 mm.

With reference to FIGS. 5 and 6 along with FIG. 4, the shell 4 in the illustrated embodiment has a slit 20 formed to extend continuously from the upper edge to the lower edge of a side surface area (right-hand side surface when viewed from the front) of the shell 4. At one side edge (left side edge in FIG. 4) of the slit 20, a protruding piece 22 extending out from an inner part in the thickness direction is formed as shown in FIG. 5. The protruding piece 22 may be in a trapezoidal shape whose upper edge extends out while inclining gradually downward, whose lower edge protrudes while inclining gradually upward, and whose leading end edge extends straightly. Advantageously, the inner surface of the protruding piece 22 is flush with the inner surface of the main portion 10, and the thickness of the protruding piece 22 is nearly a half of the thickness of the main portion 10. In the other side edge part of the slit 20 (right side part in FIG. 4), a recessed concavity 24 is formed in an inner part in the thickness direction. It is preferred that the recess depth of the recessed concavity 24 be substantially the same as the thickness of the protruding piece 22, and the contour of the recessed concavity 24 be substantially the same as the contour of the protruding piece 22 (accordingly, trapezoidal). On the face (outer surface) of the protruding piece 22 and the bottom surface of the recessed concavity 24, mutual coupling means (not shown) to be separably coupled together are arranged. Such a mutual coupling means can be composed of a coupling member marketed, for example, under the trade name “Velcro”. The cranial deformation correction helmet 2 is put on an infant's head, with the mutual coupling means being separated from each other, and then the mutual coupling means are coupled together, whereby the cranial deformation correction helmet 2 can be clamped on the infant's head at a relatively mild required pressure. If desired, in accordance with the growth of the infant's skull, the protruding piece 22 and the recessed concavity 24 can be somewhat separated in the left/right direction in FIG. 4 to increase the width of the slit 20, whereby the inner dimensions of the cranial deformation correction helmet 2 can be increased to some extent. In the illustrated embodiment, with the mutual coupling means being coupled together, regions on both sides of the slit 20 in the inner surface of the shell 4 continue smoothly, and regions on both sides of the slit 20 in the face of the shell 4 also continue smoothly, without any projections existent. Thus, the infant wearing the cranial deformation correction helmet 2 is not inhibited by the cranial deformation correction helmet 2 from rolling over while sleeping. Nor is the cranial deformation correction helmet 2 displaced to inhibit proper correction, when the infant rolls over in sleep.

It is important that the shell 4 be shaped by selective laser sintering based on the outer shape of the deformed skull to be corrected, and that its relative density be 90 to 98%, preferably 92 to 96%. If the relative density is too high, the weight of the shell is too great, but if the relative density is too low, the strength of the shell tends to be too low. The outer shape of the deformed skull to be corrected can be finalized by the three-dimensional scan mode well known per se. The selective laser sintering is a publicly known shaping method, and thus its detailed explanation will be omitted herein. Examples of the synthetic resin material for formation of the shell are relatively rigid synthetic resins such as polyamide (nylon), polycarbonate, polyester, polyacetal, polyethylene, polypropylene, polyvinyl chloride, polystyrene, polybutylene, ABS resin, cellulosic resin, acrylic resin, epoxy resin, and fluoroplastic. From the viewpoints of shapability by selective laser sintering, strength and hardness, polyamide, especially polyamide 11, is preferred. The Shore D hardness of the shell 4 shaped is preferably 70 to 85, particularly 75 to 80.

With reference to FIGS. 1 to 3, the liner 6 is formed from a foamed synthetic resin, preferably an open-cell foamed synthetic resin, and is disposed on the inner surface of the shell 4. Advantageously, the liner 6 is stuck detachably to the inner surface of the shell 4, for example, via a double-coated adhesive tape and, if contamination with sweat from the wearer of the cranial deformation correction helmet 2 proceeds, the liner 6 can be replaced as appropriate. The liner 6 is preferably disposed on all the inner surface of the shell 4, except the aforementioned thick-walled reinforcing portions 18. If desired, the liner 6 can be stuck selectively to suitable sites of the inner surface of the shell 4. The liner 6 formed from a foamed synthetic resin preferably has a thickness of 6 to 20 mm, and has a ball rebound resilience (ASTM D3574) of 1% or less. The preferred foamed synthetic resin forming the liner 6 is an open-cell foamed polyurethane, and its examples include an open-cell foamed polyurethane marketed under the trade name “Memory Foam CF-45” by K.C.C. SHOKAI LIMITED located at 1-2-1 Murotani, Nishi-ku, Kobe City, Hyogo Prefecture. The liner 6 disposed on the inner surface of the shell 4 functions as a so-called cushioning material, and functions to absorb and dissipate the sweat of the wearer. The sweat absorbed to the liner 6 is evaporated through the through-holes 16 formed in the shell 4.

### EXAMPLE 1

Using a polyamide 11 powder marketed under the trade name “ASPEX-FPA” by Aspect Inc. located at 3104-1-101, Higashinaganuma, Inagi, Tokyo, a shell of a shape as illustrated in FIGS. 1 to 6 was shaped by a selective laser sintering device marketed under the trade name “RaFaEl” by Aspect Inc. The lamination pitch was 0.1 mm. The average internal diameter of the shell was about 150 mm, the overall height was about 150 EMI, the thickness of the site excluding the thick-walled reinforcing portion was 3.0 mm, and the cross-sectional diameter of the thick-walled reinforcing portion was 6.0 mm. The average diameter of the opening in the upper surface of the shell was about 90 mm, and 82 of the through-holes with a diameter of 11.0 mm were formed in the shell. The Shore D hardness of the shell was 77, the relative density of the shell was 95%, and the total weight of the shell was 63.0 g.

The above-described shell was allowed to drop naturally in an upright state from a height of 150 cm onto a flat concrete floor. Then, the state of the shell was observed, but no damage, such as cracking, was seen in the shell.

### EXAMPLE 2

A shell was shaped in the same manner as in Example 1, except that the lamination pitch was 0.15 mm. The Shore D hardness of the shell was 77, the relative density of the shell was 94%, and the total weight of the shell was 61.5 g.

The above-described shell was allowed to drop naturally in an upright state from a height of 150 cm onto a flat concrete floor. Then, the state of the shell was observed, but no damage, such as cracking, was seen in the shell.

### EXAMPLE 3

A shell was shaped in the same manner as in Example 1, except that the lamination pitch was 0.2 mm. The Shore D hardness of the shell was 77, the relative density of the shell was 93%, and the total weight of the shell was 60.0 g.

The above-described shell was allowed to drop naturally in an upright state from a height of 150 cm onto a flat concrete floor. Then, the state of the shell was observed, but no damage, such as cracking, was seen in the shell.

### EXPLANATIONS OF LETTERS OR NUMERALS

2: Cranial deformation correction helmet

4: Outer shell

6: inner liner

16: Through-hole

18: Thick-walled reinforcing portion

20: Slit

22: Protruding piece

24: Recessed concavity

1. A cranial deformation correction helmet comprising a non-foamed synthetic resin outer shell, and a foamed synthetic resin inner liner disposed on an inner surface of the shell, wherein a relative density of the shell is 90 to 98%.

2. The cranial deformation correction helmet according to claim 1, wherein
a thick-walled reinforcing portion having an increased wall thickness is formed at an outer peripheral edge of the shell.

3. The cranial deformation correction helmet according to claim 1, wherein
a plurality of ventilation through-holes are formed in the shell.

4. The cranial deformation correction helmet according to claim 1, wherein
a Shore D hardness of the shell is 70 to 85.

5. The cranial deformation correction helmet according to claim 1, wherein
a ball rebound resilience (ASTM D3574) of the liner is 1% or less.

6. The cranial deformation correction helmet according to claim 1, wherein
the shell is opened at a site thereof corresponding to a top of a skull and is in an annular shape as a whole.

7. The cranial deformation correction helmet according to claim 6, wherein
a slit extending from an upper edge to a lower edge of the shell is formed;
a protruding piece extending out from an inner part in a thickness direction is disposed at a side edge of the slit;
a recessed concavity corresponding to the protruding piece is disposed in an inner part in the thickness direction at another side edge of the slit;
mutual coupling means to be separably coupled together are arranged on a face of the protruding piece and a bottom surface of the recessed concavity; and
when the protruding piece is positioned in the recessed concavity, and the protruding piece and the recessed concavity are coupled together by the mutual coupling means, regions on both sides of the slit on a surface of the shell continue smoothly.

8. A method for producing the cranial deformation correction helmet according to claim 1, comprising:
shaping the shell by selective laser sintering based on an outer shape of a skull to be corrected; and
then disposing the liner on the inner surface of the shell.
